(19) United States
(12) Patent Application Publication
(10) Pub. No.: US 2024/0287521 A1
(43) Pub. Date: Aug. 29, 2024

(54) Allele-specific silencing therapy for DFNA21 using antisense oligonucleotides

(71) Applicant: Stichting Radboud universitair medisch centrum, Nijmegen (NL)

(72) Inventors: Hendrikus Antonius Rudolfus Van Wyk, Nijmegen (NL); Johanna Maria Josephina Kremer, Oostrum (NL); Erik De Vrieze, Nijmegen (NL); Suzanne De Bruijn, Nijmegen (NL)

(73) Assignee: Stichting Radboud universitair medisch centrum, Nijmegen (NL)

(21) Appl. No.: 18/571,755

(22) PCT Filed: Jun. 24, 2022
(86) PCT No.: PCT/EP2022/067298

§ 371 Date: Dec. 19, 2023

(30) Foreign Application Priority Data
Jun. 25, 2021 (EP) 21181836.4

(51) Int. Cl. C12N 15/113

(52) U.S. Cl. CPC C12N 15/113; C12N 2310/11; C12N 2310/321; C12N 2310/341; C12N 2320/34

(57) ABSTRACT

The invention relates to the fields of medicine and immunology. In particular, it relates to novel antisense oligonucleotides that may be used in the treatment, prevention and/or delay of an RIPOR2 associated condition.

### FIELD OF THE INVENTION

The invention relates to the fields of medicine and immunology. In particular, it relates to novel antisense oligonucleotides that may be used in the treatment, prevention and/or delay of conditions associated with genetic variants in RIPOR2.

### BACKGROUND OF THE INVENTION

Hearing loss is one of the most prevalent disabilities worldwide, and has a significant impact on quality of life. The adult-onset type of the condition is highly heritable but the genetic causes are largely unknown, which is in contrast to childhood-onset hearing loss. DFNA21, caused by an in-frame deletion in the RIPOR2 gene, is a progressive form of dominantly inherited hearing loss (HL). The RIPOR2 gene is located on chromosome 6 (chr6:24,804,282-24,936,052 [GRCh38/hg38], NCBI reference sequence NG_051606.1). Recently, the in-frame deletion (c.1696_1707del, p.(Gln566_Lys569del), NM_014722.3; SEQ ID NO: 1) was identified as the most frequent cause of this type of HL within Europe. It is estimated that >30,000 individuals within Northwest Europe carry this variant, and therefore are at risk to develop DFNA21.[1] DFNA21 is highly variable; the age of onset ranges from congenital to the 7^{th} decade of life, and HL can progress from mild in early stages of the disease, to severe or profound later in life.[1-3] Currently, there is no cure available to treat DFNA21.

RIPOR2 encodes the RHO Family Interacting Cell Polarisation Regulator 2 protein (RIPOR2). The protein is expressed in a wide variety of tissues and cell types, including the cochlea. [4] In mouse cochlear hair cells, RIPOR2 is described to be localized at the basal taper region of stereocilia, where it is organized in a ring-like fashion. In the absence of RIPOR2, development, function and maintenance of murine hair cells is severely affected and morphological defects in both hair cells and stereociliary structures can be observed. Ripor2 knockout mice are already deaf at 4 weeks of age, and ripor2 knockdown in zebrafish leads to profound HL and loss of saccular hair cells.[4-6]

Pathogenic variants in RIPOR2 have not only been associated with dominantly inherited, but with recessively inherited HL (DFNB104) as well.[6] A loss-of-function variant in exon 3 of the gene is associated with profound, prelingual HL that corresponds with the phenotypes observed in the mouse knockout and zebrafish knockdown models. [4,6] The c.1696_1707del variant that affects exon 14, on the other hand, can be considered a milder variant and is associated with a less severe phenotype, with a later onset. [1] The aberrant localization of mutant RIPOR2 in the stereocilia of wildtype mouse cochlear hair cells, and the inability to rescue morphological defects of the hair bundle of RIPOR2-deficient hair cells, confirm the pathogenicity of this variant. Although the pathogenic mechanism of the in-frame deletion is not yet completely understood, there are strong indications that the variant acts via a non-haploinsufficiency mechanism. The mislocalization of mutant RIPOR2 in the stereocilia suggests a toxic gain-of-function effect. In line with this, both humans and mice carrying heterozygous loss-of-function alleles do not display HL[4,6]

The non-haploinsufficiency disease mechanism suggests that inhibiting the synthesis of mutant RIPOR2 proteins or degrading the mutant RIPOR2 proteins, can alleviate the effect of mutant RIPOR2 on auditory functions. However, to date, no compounds or solutions, capable of targeting the synthesis of mutant RIPOR2 proteins or the proteins themselves have been described.

Antisense oligonucleotides (AONs) with DNA-like properties can be specifically designed to bind transcripts harboring pathogenic variants, and will subsequently recruit RNase H1 endonuclease. This endonuclease degrades RNA molecules that are part of RNA:DNA duplexes, which will lead to a sequence-specific decrease in protein synthesis.[7,8] The 5′ and 3′ wings of the RNase H1-dependent AONs can be chemically modified to increase thermodynamic stability and nuclease resistance, while maintaining a central gap region of DNA nucleotides that ensures RNase H1 activity.[9] These modified AONs are referred to as gapmers, and have shown great therapeutic potential in treatment strategies for other inherited disorders including amyotrophic lateral sclerosis (ALS) and Huntington disease.[10] There are only few reports of therapeutic application of AONs in the inner ear, most of which focus on splice-correction therapy. WO2021/084021 for example, describes AONs capable of degrading (pre-)mRNAs transcribed from the mutated COCH gene. Mutations in COCH cause DFNA9 a common form of inherited progressive hearing loss and vestibular dysfunction. To date, there are four AON-gapmers on the market that have been FDA- or EMA-approved and many more gapmers are under investigation in clinical trials.[12]

The fact that the c.1696_1707del variants in RIPOR2 is currently the only identified genetic cause for the DFNA21-type of hearing loss, and the estimated 30,000 Europeans carrying this mutation, render it an attractive target for antisense oligonucleotide (AON)-based therapy. Accordingly, there is an urgency to develop AONs with the purpose to degrade RIPOR2 c.1696_1707del transcripts to prevent the formation of mutant RIPOR2 proteins to delay or halt disease progression or prevent hearing impairment altogether.

### SUMMARY OF THE INVENTION

The invention relates to an antisense oligonucleotide (AON) moiety for the degradation of a mutated RIPOR2 transcript that binds to and/or is complementary to a polynucleotide with the nucleotide sequence as set forward in SEQ ID NO: 1. Preferably the AON binds to or is complementary to a polynucleotide part within SEQ ID NO: 1, said polynucleotide part having a nucleotide sequence selected from the group consisting of SEQ ID NO: 2, 3, and 4.

In a further aspect, the invention relates to a pharmaceutical composition comprising an AON as described hereon for the degradation of a mutated RIPOR2 and further comprising a pharmaceutically acceptable excipient.

In another aspect, the invention relates to the AON for the degradation of a mutated RIPOR2 as described herein or the pharmaceutical composition according as described herein for use as a medicament. Preferably for use as a medicament for treating a RIPOR2 related disease or a condition requiring the degradation of mutated RIPOR2 (pre)mRNA.

In yet another aspect, the invention relates to a use of the AON for the degradation of a mutated RIPOR2 as described herein or the pharmaceutical composition according as described herein for treating a RIPOR2 related disease or a condition requiring the degradation of mutated RIPOR2 (pre)mRNA.

The invention further relates to a method of treatment of a RIPOR2 related disease or condition requiring the degradation of mutated RIPOR2 (pre)mRNA in a subject in need thereof, comprising administration of an antisense oligonucleotide for the degradation of a mutated RIPOR2 as defined herein. In certain embodiments, the RIPOR2 related disease or condition is a condition resulting in hearing impairment and/or vestibular dysfunction, preferably wherein the condition is a vestibulo-cochlear disorder, more preferably wherein the disease or condition is DFNA21.

The invention also relate to a method for the degradation of a mutated RIPOR2 in a cell, the method comprising contacting the cell with an antisense oligonucleotide for the degradation of a mutated RIPOR2 as defined herein or the pharmaceutical composition as defined herein.

### DETAILED DESCRIPTION OF THE INVENTION

By definition, antisense oligonucleotides (AONs) are substantially complementary (i.e. antisense) to their target, allowing them to bind to the corresponding pre-mRNA molecule. On the basis of mechanism of action, two main classes of antisense oligonucleotide can generally be discerned: (a) the RNase H-dependent oligonucleotides, which induce degradation of mRNA; and (b) the steric-blocker oligonucleotides, which physically prevent or inhibit progression of splicing or the translational machinery. The inventors have identified AONs that allow the degradation of mutant RIPOR2 transcripts that are associated with DFNA21.

Accordingly, in a first aspect the invention provides for an antisense oligonucleotide moiety for the degradation of a mutated RIPOR2 transcript that binds to and/or is complementary to a polynucleotide with the nucleotide sequence as set forward in SEQ ID NO: 1. Preferably the antisense oligonucleotide moiety binds to or is complementary to a polynucleotide part within SEQ ID NO: 1, said polynucleotide part having a nucleotide sequence selected from the group consisting of SEQ ID NO: 2, 3, and 4.

The terms “antisense oligonucleotide” and “AON” are used interchangeably herein and are understood to refer to an oligonucleotide molecule comprising a nucleotide sequence which is substantially complementary to a target nucleotide sequence in a pre-mRNA molecule, hnRNA (heterogenous nuclear RNA) or mRNA molecule. The degree of complementarity (or substantial complementarity) of the antisense sequence is preferably such that a molecule comprising the antisense sequence can form a stable hybrid with the target nucleotide sequence in the RNA molecule under physiological conditions. Binding of an AON to its target can easily be assessed by the person skilled in the art using techniques that are known in the field such as the gel mobility shift assay as described in EP1619249.

The term “complementary” used in the context of the invention indicates that some mismatches in the antisense sequence are allowed as long as the functionality, i.e. promoting mRNA degradation is achieved. Preferably, the complementarity is from 90% to 100%. In general this allows for 1 or 2 mismatches in an AON of 20 nucleotides or 1, 2, 3 or 4 mismatches in an AON of 40 nucleotides, or 1, 2, 3, 4, 5 or 6 mismatches in an AON of 60 nucleotides, etc. Optionally, said AON may further be tested by transfection into isolated cells from patients having a mutated RIPOR2. The complementary regions are preferably designed such that, when combined, they are specific for the intron or exon in the pre-mRNA or mRNA. Such specificity may be created with various lengths of complementary regions, as this depends on the actual sequences in other (pre-)mRNA molecules in the system. The risk that the AON will also be able to hybridize to one or more other (pre-)mRNA molecules decreases with increasing size of the AON. It is clear that AONs comprising mismatches in the region of complementarity but that retain the capacity to hybridize and/or bind to the targeted region(s) in the (pre-)mRNA, can be used in the invention. However, preferably at least the complementary parts do not comprise such mismatches as AONs lacking mismatches in the complementary part typically have a higher efficiency and a higher specificity than AONs having such mismatches in one or more complementary regions. It is thought, that higher hybridization strengths, (i.e. increasing number of interactions with the opposing strand) are favorable in increasing the efficiency of the process of interfering with the splicing or mRNA degradation machinery of the system.

In certain embodiments, the degradation is nuclease degradation (e.g., RNase H).

The most effective AON-based therapy in the treatment of dominantly inherited non-haploinsufficiency disorder ensures that protein production from the healthy allele remains unaffected. The mutant and healthy RIPOR2-encoding sequence differ from each other by 12 nucleotides. Therefore, in a preferred embodiment, the antisense oligonucleotide of the invention is an AON that specifically targets the mutated allele and preferably does not target the wild-type allele.

In certain embodiments the AONs according to the invention cause specific degradation of the mutated RIPOR2 allele and do not degrade will-type RIPOR2 allele i.e. in certain embodiments, the AONs according to the invention are able to discriminate between mutated RIPOR2 and wild-type RIPOR2. Therefore in certain embodiments, the invention relates to AONs for the specific degradation of mutated RIPOR2.

The AON according to the invention preferably does not contain a stretch of CpG, more preferably does not contain any CpG. The presence of a CpG or a stretch of CpG in an oligonucleotide is usually associated with an increased immunogenicity of said oligonucleotide (Dorn and Kippenberger, 2008). This increased immunogenicity is undesired since it may induce damage of the tissue to be treated, i.e. the inner ear. Immunogenicity may be assessed in an animal model by assessing the presence of CD4+ and/or CD8+ cells and/or inflammatory mononucleocyte infiltration. Immunogenicity may also be assessed in blood of an animal or of a human being treated with an AON according to the invention by detecting the presence of a neutralizing antibody and/or an antibody recognizing said AON using a standard immunoassay known to the skilled person. An inflammatory reaction, type I-like interferon production, IL-12 production and/or an increase in immunogenicity may be assessed by detecting the presence or an increasing amount of a neutralizing antibody or an antibody recognizing said AON using a standard immunoassay. The AON according to the invention furthermore preferably has acceptable RNA binding kinetics and/or thermodynamic properties. The RNA binding kinetics and/or thermodynamic properties are at least in part determined by the melting temperature of an oligonucleotide (Tm; calculated with the oligonucleotide properties calculator (www.unc.edu/-cail/biotool/oligo/index) for single stranded RNA using the basic Tm and the nearest neighbor model), and/or the free energy of the AON-target intron/exon complex (using RNA structure version 4.5). If a Tm is too high, the AON is expected to be less specific. An acceptable Tm and free energy depend on the sequence of the AON. Therefore, it is difficult to give preferred ranges for each of these parameters. An acceptable Tm may be ranged between 35 and 70° C. and an acceptable free energy may be ranged between 15 and 45 kcal/mol. In all embodiments, the nucleotide in the antisense oligonucleotide according to the invention may be, wherein a nucleotide in the antisense oligonucleotide may be an RNA residue, a DNA residue, and/or a nucleotide analogue or equivalent. Preferably, the antisense oligonucleotide comprises both RNA and DNA residues. More preferably, the antisense oligonucleotide as described herein is a GapmeR.

“GapmeR” or “gap oligomer”, as used herein, refers to a chimeric oligomer having a central portion (a “gap”) flanked by 3′ and 5′ “wings”, wherein the gap has a modification that is different as compared to each of the wings. Such modifications may include nucleobase, monomeric linkage, and sugar modifications as well as the absence of a modification (such as unmodified RNA or DNA). Accordingly, a gapmer may be as simple as RNA wings separated by a DNA gap. In some cases, the nucleotide linkages in the wings may be different than the nucleotide linkages in the gap. In certain embodiments, each wing comprises nucleotides with high affinity modifications and the gap comprises nucleotides that do not comprise that modification.

Alternatively, the nucleotides in the gap and the nucleotides in the wings may have high affinity modifications, but the high affinity modifications in the gap are different than the high affinity modifications in each of the wings. The modifications in the wings may confer resistance to cleavage by endogenous nucleases, including RNaseH, while the modifications in the gap may be substrates for RNase H. The modifications in the wings may confer resistance to cleavage by endogenous nucleases, including RNaseH, while the modifications in the gap may be substrates for RNase H. The modifications in the wings may be the same or different from one another. The nucleotides in the gap may be unmodified and nucleotides in the wings may be modified.

A GapmeR has a wing-gap-wing ratio, which may be represented numerically (wing #-gap #-wing #). The GapmeR may be symmetrical for example 7-12-7, 7-11-7, 7-10-7, 7-9-7, 7-8-7, 7-7-7, 7-6-7, 7-5-7, 7-4-7, 7-3-7, 6-12-6, 6-11-6, 6-10-6, 6-9-6, 6-8-6, 6-7-6, 6-6-6, 6-5-6, 6-4-6, 6-3-6, 6-2-6, 5-12-5, 5-11-5, 5-10-5, 5-9-5, 5-8-5, 5-7-5, 5-6-5, 5-5-5, 5-4-5, 5-3-5, 4-12-4, 4-11-4, 4-10-4, 4-9-4, 4-8-4, 4-7-4, 4-6-4, 4-5-4, 4-4-4, 4-3-4, 3-12-3, 3-11-3, 3-10-3, 3-9-3, 3-8-3, 3-7-3, 3-6-3, 3-5-3, or 3-4-3.

In one embodiment, the Gapmer may be asymmetrical for example, 8-13-9, 8-12-9, 8-11-9, 8-10-9, 8-9-9, 8-8-9, 8-7-9, 8-6-9, 8-5-9, 8-4-9, 8-3-9, 8-2-9, 8-1-9, 7-15-8, 7-14-8, 7-13-8, 7-12-8, 7-11-8, 7-10-8, 7-9-8, 7-8-8, 7-7-8, 7-6-8, 7-5-8, 7-4-8, 7-3-8, 7-2-8, 7-1-8, 6-15-7, 6-14-7, 6-13-7, 6-12-7, 6-11-7, 6-10-7, 6-9-7, 6-8-7, 6-7-7, 6-6-7, 6-5-7, 6-4-7, 6-3-7, 6-2-7, 6-1-7, 5-15-6, 5-14-6, 5-13-6, 5-12-6, 5-11-6, 5-10-6, 5-9-6, 5-8-6, 5-7-6, 5-6-6, 5-5-6, 5-4-6, 5-3-6, 5-2-6, 5-1-6, 4-15-5, 4-14-5, 4-13-5, 4-13-3, 4-12-5, 4-11-5, 4-10-5, 4-9-5, 4-8-5, 4-7-5, 4-6-5, 4-5-5, 4-4-5, 4-3-5, 4-2-5, 4-1-5, 3-17-4, 3-16-4, 3-15-4, 3-14-4, 3-13-4, 3-12-4, 3-11-4, 3-10-4, 3-9-4, 3-8-4, 3-7-4, 3-6-4, 3-5-4, 3-4-4, 3-3-4, 2-24-3, 2-23-3, 2-22-3, 2-21-3, 2-20-3, 2-19-3, 2-18-3, 2-17-3, 2-16-3, 2-15-3, 2-14-3, 2-13-3, 2-12-3, 2-11-3, 2-10-3, 2-9-3, 2-8-3, 2-7-3, 2-6-3, 2-5-3, 2-4-3, 1-26-2, 1-25-2, 1-24-2, 1-22-2, 1-21-2, 1-20-2, 1-19-2, 1-18-2, 1-17-2, 1-16-2, 1-15-2, 1-14-2, 1-13-2, 1-12-2, 1-11-2, 1-10-2, 1-9-2, 1-8-2, 1-7-2, 3-26-1, 3-5-1, 3-24-1, 3-22-1, 3-21-1, 3-20-1, 3-19-1, 3-18-1, 3-17-1, 3-16-1, 3-15-1, 3-14-1, 4-13-1, 4-12-1, 4-11-1, 4-10-1, 3-9-1, 3-8-1 or 4-7-1. Preferably the gapmer is asymmetrical and has a 7-10-3 structure.

A preferred AON for the degradation of a mutated RIPOR2 according to the invention, has a length of from about 8 to about 40 nucleotides, preferably from about 10 to about 40 nucleotides, more preferably from about 14 to about 30 nucleotides, more preferably from about 16 to about 24 nucleotides, such as 16, 17, 18, 19, 20, 21, 22, 23 or 24 nucleotides. Preferably, an AON according to the invention has a length of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. Most preferably, the AON has a length of about 20 nucleotides.

A preferred AON for the degradation of a mutated RIPOR2 according to the invention comprises or consists of an oligonucleotide with the sequence as set forward in SEQ ID NO: 5, 6, 7, 8, 9, 10, 11 and 21.

In an embodiment, the AON as described herein is complementary to a polynucleotide with the nucleotide sequence as set forward in SEQ ID NO: 2 and the preferred AONs comprise or consist of a sequence selected from the group consisting of SEQ ID NO: 5, 6, 7, 8, 9, 10, 11 and 21.

In an embodiment, the AON as described herein is complementary to a polynucleotide with the nucleotide sequence as set forward in SEQ ID NO: 3 and the preferred AONs comprise or consist of a sequence selected from the group consisting of SEQ ID NO: 5, 6, 7, 8, 9, 10, 11 and 21.

In an embodiment, the AON as described herein is complementary to a polynucleotide with the nucleotide sequence as set forward in SEQ ID NO: 4 and the preferred AONs comprise or consist of a sequence selected from the group consisting of SEQ ID NO: 5, 6, 7, 8, 9, 10, 11 and 21.

It is preferred that an AON for the degradation of a mutated RIPOR2 according to the invention comprises one or more residues that are modified to increase nuclease resistance, and/or to increase the affinity of the antisense oligonucleotide for the target sequence. Therefore, in a preferred embodiment, the AON comprises at least one nucleotide analogue or equivalent, wherein a nucleotide analogue or equivalent is defined as a residue having a modified base, and/or a modified backbone, and/or a non-natural internucleoside linkage, or a combination of these modifications.

In a preferred embodiment, the nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Most preferably, the AON according to the invention comprises a phosphorothiorate backbone.

Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells. A recent report, demonstrated triplex formation by a morpholino oligonucleotide and, because of the non-ionic backbone, these studies showed that the morpholino oligonucleotide was capable of triplex formation in the absence of magnesium.

It is further preferred that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen et al., 1991). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar, 2005). Since the backbone of a PNA molecule contains no charged phosphate groups, PNA-RNA hybrids are usually more stable than RNA-RNA or RNA-DNA hybrids, respectively (Egholm et al., 1993). A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage. In yet a further embodiment, a nucleotide analogue or equivalent according to the invention comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent according to the invention comprises one or more sugar moieties that are mono- or disubstituted at the 2′, 3′ and/or 5′ position such as a —OH; —F; substituted or unsubstituted, linear or branched lower (Cl—C10) alkyl, alkenyl, alkynyl, alkaryl, allyl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; methoxyethoxy; dimethylaminooxyethoxy; and -dimethylaminoethoxyethoxy. The sugar moiety can be a pyranose or derivative thereof, or a deoxypyranose or derivative thereof, preferably ribose or derivative thereof, or deoxyribose or derivative of. A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-0, 4′-C-ethylene-bridged nucleic acid (Morita et al., 2001). These substitutions render the nucleotide analogue or equivalent RNase H and nuclease resistant and increase the affinity for the target RNA. In another embodiment, a nucleotide analogue or equivalent according to the invention comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

It is understood by a skilled person that it is not necessary for all positions in an AON to be modified uniformly. In addition, more than one of the aforementioned analogues or equivalents may be incorporated in a single AON or even at a single position within an AON. In certain embodiments, an AON according to the invention has at least two different types of analogues or equivalents. Accordingly, in a preferred embodiment an antisense oligonucleotide for the degradation of a mutated RIPOR2 according to the invention, comprises a 2′-0 alkyl phosphorothioate antisense oligonucleotide, such as 2′-O-methyl modified ribose (RNA), 2′-O-ethyl modified ribose, 2′-O-propyl modified ribose, 2-0′-methoxy ethyl-ribose and/or substituted derivatives of these modifications such as halogenated derivatives. In certain preferred embodiments, an AON according to the invention comprises an 8-Oxoguanine (8-oxoG) modification. An 8-oxoG modification, is an oxidized version of a regular guanine (G) that has a reduced affinity for cytosine (C), but can form base-pairs with adenine (A).

Preferably, when the AONs according to the invention is a gapmer, the gapmer is modified to comprise a phosphorothioate backbone and a 2′-O-methyl modified ribose.

It will also be understood by the skilled person that different antisense oligonucleotides can be combined for the specific degradation of a mutated RIPOR2. Accordingly, the invention provides for a set of antisense oligonucleotide for promoting specific degradation of RIPOR2 comprising at least two antisense oligonucleotides as defined herein.

An AON for the specific degradation of a mutated RIPOR2 to the invention may be indirectly administrated using suitable means known in the art. It may for example be provided to an individual or a cell, tissue or organ of said individual as such, as a so-called ‘naked’ AON. It may also be administered in the form of an expression vector wherein the expression vector encodes an RNA transcript comprising the sequence of said AON according to the invention. The expression vector is preferably introduced into a cell, tissue, organ or individual via a gene delivery vehicle. In a preferred embodiment, when the AON comprises or consists of unmodified RNA residues, there is provided a viral-based expression vector comprising an expression cassette or a transcription cassette that drives expression or transcription of an AON according to the invention. Accordingly, the invention provides for a viral vector expressing antisense oligonucleotide according to the invention when placed under conditions conducive to expression of the antisense oligonucleotide.

A cell can be provided with an AON for the specific degradation of a mutated RIPOR2 to the invention by plasmid-derived antisense oligonucleotide expression or viral expression provided by adenovirus- or adeno-associated virus-based vectors. Expression may be driven by an RNA polymerase II promoter (Pol II) such as a U7 RNA promoter or an RNA polymerase III (Pol III) promoter, such as a U6 RNA promoter. A preferred delivery vehicle is a viral vector such as an adeno-associated virus vector (AAV), or a retroviral vector such as a lentivirus vector and the like. Also, plasmids, artificial chromosomes, plasmids usable for targeted homologous recombination and integration in the human genome of cells may be suitably applied for delivery of an AON according to the invention. Preferred for the invention are those vectors wherein transcription is driven from Pol III promoters, and/or wherein transcripts are in the form fusions with U1 or U7 transcripts, which yield good results for delivering small transcripts. It is within the skill of the artisan to design suitable transcripts. Preferred are Pol III driven transcripts, preferably, in the form of a fusion transcript with an U1 or U7 transcript. Such fusions may be generated as previously described (Gorman et al., 1998).

A preferred expression system for an AON for specific degradation of a mutated RIPOR2 according to the invention is an adenovirus associated virus (AAV)-based vector. Single chain and double chain AAV-based vectors have been developed that can be used for prolonged expression of antisense nucleotide sequences for highly efficient degradation of transcripts. A preferred AAV-based vector, for instance, comprises an expression cassette that is driven by an RNA polymerase III-promoter (Pol III) or an RNA polymerase II promoter (Pol II). A preferred RNA promoter is, for example, a Pol III U6 RNA promoter, or a Pol II U7 RNA promoter.

The invention accordingly provides for a viral-based vector, comprising a Pol II or a Pol III promoter driven expression cassette for expression of an AON for the specific degradation of a mutated RIPOR2 according to the invention.

An AAV vector according to the invention is a recombinant AAV vector and refers to an AAV vector comprising part of an AAV genome comprising an encoded AON for the specific degradation of a mutated RIPOR2 according to the invention encapsidated in a protein shell of capsid protein derived from an AAV serotype as depicted elsewhere herein. Part of an AAV genome may contain the inverted terminal repeats (ITR) derived from an adeno-associated virus serotype, such as AAV1, AAV2, AAV3, AAV4, AAV5, AAV8, AAV9 and others. A protein shell comprised of capsid protein may be derived from an AAV serotype such as AAV1, 2, 3, 4, 5, 8, 9 and others. A protein shell may also be named a capsid protein shell. AAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV5 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention.

Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV2/5, AAV2/8, AAV2/9, AAV5/2, AAV5/5, AAV5/8, AAV5/9, AAV8/2, AAV8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV2/5 vector.

More preferably, a recombinant AAV vector according to the invention comprises a capsid protein shell of AAV serotype 2 and the AAV genome or ITRs present in said vector are derived from AAV serotype 8; such vector is referred to as an AAV2/8 vector.

More preferably, a recombinant AAV vector according to the invention comprises a capsid protein shell of AAV serotype 2 and the AAV genome or ITRs present in said vector are derived from AAV serotype 9; such vector is referred to as an AAV2/9 vector.

More preferably, a recombinant AAV vector according to the invention comprises a capsid protein shell of AAV serotype 2 and the AAV genome or ITRs present in said vector are derived from AAV serotype 2; such vector is referred to as an AAV2/2 vector.

A nucleic acid molecule encoding an AON according to the invention represented by a nucleic acid sequence of choice is preferably inserted between the AAV genome or ITR sequences as identified above, for example an expression construct comprising an expression regulatory element operably linked to a coding sequence and a 3′ termination sequence.

“AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art, see e.g. (Chiorini et al., 1999) or U.S. Pat. No. 5,139,941, incorporated herein by reference. The AAV helper functions can be supplied on an AAV helper construct, which may be a plasmid. Introduction of the helper construct into the host cell can occur e.g. by transformation, transfection, or transduction prior to or concurrently with the introduction of the AAV genome present in the AAV vector as identified herein. The AAV helper constructs according to the invention may thus be chosen such that they produce the desired combination of serotypes for the AAV vector's capsid protein shell on the one hand and for the AAV genome present in said AAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference.

Preferably, an AAV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

A preferred AAV vector according to the invention is an AAV vector, preferably an AAV2/5, AAV2/8, AAV2/9 or AAV2/2 vector, carrying an AON for promoting mRNA degradation according to the invention that is an AON that comprises, or preferably consists of, a sequence that is: complementary or substantially complementary to a nucleotide sequence consisting of SEQ ID NO 1, preferably the antisense oligonucleotide moiety binds to or is complementary to a polynucleotide part within SEQ ID NO: 1 with a nucleotide sequence selected from the group consisting of SEQ ID NO: 2, 3 and 4.

Even more preferably, the AON comprises or consists of a polynucleotide with a nucleotide sequence selected from the group consisting of SEQ ID NO: 5, 6, 7, 8, 9, 10, 11 and 21. Improvements in means for providing an individual or a cell, tissue, organ of said individual with an AON for promoting the degradation of mutated RIPOR2 mRNA according to the invention, are anticipated considering the progress that has already thus far been achieved. Such future improvements may of course be incorporated to achieve the mentioned effect on restructuring of mRNA using a method according to the invention.

Alternatively, a preferred delivery method for an AON the specific degradation of a mutated RIPOR2 as described herein or a plasmid for expression of such AON is a viral vector or are nanoparticles. In certain embodiments, the preferred delivery method for an AON as described herein is by use of slow-release or sustained release capsules. In certain embodiments, the preferred delivery method for an AON as described herein is by use of hydrogels (such as described in WO1993/01286).

Alternatively, a plasmid can be provided by transfection using known transfection agents. For intravenous, subcutaneous, intratympanic, nasal, intramuscular, intrathecal and/or intraventricular administration it is preferred that the solution is a physiological salt solution. Particularly preferred in the invention is the use of an excipient or transfection agents that will aid in delivery of each of the constituents as defined herein to a cell and/or into a cell, preferably a cell expressing the mutated RIPOR2. Preferred are excipients or transfection agents capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection agentia comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphiles (SAINT-18), Lipofectin™, DOTAP and/or viral capsid proteins that are capable of self-assembly into particles that can deliver each constituent as defined herein to a cell, preferably a cell that expresses mutated RIPOR2. Such excipients have been shown to efficiently deliver an oligonucleotide such as AONs to a wide variety of cultured cells in vitro, including RIPOR2-expressing T-REx 293 cells. The ease of structural modification can be used to allow further modifications and the analysis of their further (in vivo) nucleic acid transfer characteristics and toxicity. Lipofectin represents an example of a liposomal transfection agent. It consists of two lipid components, a cationic lipid N-[1-(2,3 dioleoyloxy)propyl]-N, N, N-trimethylammonium chloride (DOTMA) (cp. DOTAP which is the methylsulfate salt) and a neutral lipid dioleoylphosphatidylethanolamine (DOPE). The neutral component mediates the intracellular release. Another group of delivery systems are polymeric nanoparticles. Polycations such as diethylaminoethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver each constituent as defined herein, preferably an AON according to the invention, across cell membranes into cells.

In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an oligonucleotide. The skilled person may select and adapt any of the above or other commercially available alternative excipients and delivery systems to package and deliver an AON for use in the current invention to deliver it for the prevention, treatment or delay of a RIPOR2 related disease or a condition requiring the degradation of mutated RIPOR2 (pre)mRNA. “Prevention, treatment or delay of an RIPOR2 related disease or a condition related disease or condition” is herein preferably defined as preventing, halting, ceasing the progression of, or (partially) reversing the formation of cytotoxic cochlin dimers.

In addition, an AON according to the invention could be covalently or non-covalently linked to a targeting ligand specifically designed to facilitate the uptake into the cell, cytoplasm and/or its nucleus. Such ligand could comprise (i) a compound (including but not limited to peptide(-like) structures) recognizing cell, tissue or organ specific elements facilitating cellular uptake and/or (ii) a chemical compound able to facilitate the uptake in to cells and/or the intracellular release of an oligonucleotide from vesicles, e.g. endosomes or lysosomes.

Therefore, in a preferred embodiment, an AON for the degradation of a mutated RIPOR2 transcript according to the invention is formulated in a composition or a medicament or a composition, which is provided with at least an excipient and/or a targeting ligand for delivery and/or a delivery device thereof to a cell and/or enhancing its intracellular delivery.

It is to be understood that if a composition comprises an additional constituent such as an adjunct compound as later defined herein, each constituent of the composition may not be suitably formulated in one single combination or composition or preparation. Depending on their identity and specific features, the skilled person will know which type of formulation is the most appropriate for each constituent as defined herein. In a preferred embodiment, the invention provides a composition or a preparation which is in the form of a kit of parts comprising an AON for the degradation of a mutated RIPOR2 according to the invention and a further adjunct compound as later defined herein.

If required and/or if desired, an AON for the degradation of a mutated RIPOR2 transcript, a set of antisense oligonucleotides according to the invention, or a vector, preferably a viral vector, according to the invention, carrying naked AONs or expressing an AON for degrading the mutated RIPOR2 transcript according to the invention can be incorporated into a pharmaceutically active mixture by adding a pharmaceutically acceptable carrier.

Accordingly, the invention also provides for a composition, preferably a pharmaceutical composition comprising an antisense oligonucleotide for the specific degradation of a mutated RIPOR2 transcript, according to the invention and a pharmaceutically acceptable excipient Such composition may comprise a single AON for degrading mutated RIPOR2 transcripts according to the invention, but may also comprise multiple, distinct AONs as described herein. Such a pharmaceutical composition may comprise any pharmaceutically acceptable excipient, including a carrier, filler, preservative, adjuvant, solubilizer and/or diluent. Such pharmaceutically acceptable carrier, filler, preservative, adjuvant, solubilizer and/or diluent may for instance be found in Remington, 2000. Each feature of said composition has earlier been defined herein.

A preferred route of administration is administration into the inner ear (intratympanic). More preferred is administration into the cochlea and/or into the vestibular organ. In one embodiment, the route of administration is nasal delivery. In certain embodiments, the AON is administered via the systemic route for example via the intravenous, subcutaneous, intramuscular, epidural or oral route.

A preferred AON for the specific degradation of a mutated RIPOR2 transcript according to the invention, is for the treatment of an RIPOR2 related disease or condition of an individual. In all embodiments of the invention, the term “treatment” is understood to include the prevention and/or delay of the RIPOR2 related disease or a condition requiring degradation of the (pre)-mRNA of RIPOR2 or promoting degradation of mutated RIPOR2 mRNA. An individual, which may be treated using an AON according to the invention may already have been diagnosed as having an RIPOR2-related disease or condition, such as DFNA21.

Alternatively, an individual which may be treated using an AON according to the invention may not have yet been clinically diagnosed as having a RIPOR2-related disease or condition requiring the degradation of RIPOR2 but may be an individual having an increased risk of developing a RIPOR2-related disease or condition, such as DFNA21 in the future given his or her genetic background. A preferred individual is a human being. In all embodiments of the invention, the RIPOR2-related disease or condition preferably is a condition resulting in hearing impairment, preferably wherein the condition is a cochlear disorder, more preferably wherein the disease or condition is DFNA21.

Accordingly, the invention further provides for an antisense oligonucleotide according to the invention, a set of antisense oligonucleotides according to the invention, or a viral vector according to the invention, or a (pharmaceutical) composition according to the invention for use as a medicament, preferably as a medicament for the treatment of an RIPOR2-related disease or condition requiring the degradation of a mutated RIPOR2 and for use as a medicament for the prevention, treatment or delay of an RIPOR2-related disease or condition requiring degradation of a mutated RIPOR2 transcript. Each feature of all medical use embodiment herein has earlier been defined herein and is preferably such feature as earlier defined herein.

The invention further provides for the use of an AON to the invention, a set of antisense oligonucleotides according to the invention, a vector according to the invention or a (pharmaceutical) composition according to the invention for treating an RIPOR2-related disease or condition requiring the degradation of mutated RIPOR2 transcript. Each feature of all medical use embodiment herein has earlier been defined herein and is preferably such feature as earlier defined herein.

The invention further provides for, a method of treatment of an RIPOR2-related disease or condition requiring degradation of a mutated RIPOR2 transcript, said method comprising contacting a cell of said individual with an AON as described herein, a vector according as described herein or a (pharmaceutical) composition as described herein. Each feature of all medical use embodiment herein has earlier been defined herein and is preferably such feature as earlier defined herein.

The invention further provides for the use of an AON as described herein, a set of antisense oligonucleotides according to the invention, a vector according to the invention or a (pharmaceutical) composition according to the invention for the preparation of a medicament for the treatment of an RIPOR2-related disease or condition requiring the specific degradation of RIPOR2 transcripts. Each feature of all medical use embodiment herein has earlier been defined herein and is preferably such feature as earlier defined herein.

The invention further provides for an antisense oligonucleotide as described herein, a set of antisense oligonucleotides as described herein, the use as described herein or the method according as described herein, wherein the RIPOR2 related disease or condition requiring degradation of a mutated RIPOR2 is a condition resulting in hearing impairment, preferably wherein the condition is a cochlear disorder, more preferably wherein the disease or condition is DFNA21.

Treatment in a use or in a method according to the invention is preferably at least once, and preferably lasts at least one week, one month, several months, one year, 2, 3, 4, 5, 6 years or longer, such as life-long. Each AON as defined herein for use according to the invention may be suitable for direct administration to a cell, tissue and/or an organ in vivo of individuals already affected or at risk of developing an RIPOR2 related disease or condition requiring degradation of mutated RIPOR2 transcripts, such as cochlear disorder or DFNA21, and may be administered directly in vivo, ex vivo or in vitro. The frequency of administration of an AON, composition, compound or adjunct compound according to the invention may depend on several parameters such as the severity of the disease, the age of the patient, the mutation of the patient, the number of AON according to the invention (i.e. dose), the formulation of the AON, composition, stability of the AON, binding affinity of the AON to the target, compound or adjunct compound according to the invention, the route of administration and so forth. The frequency of administration may vary between daily, weekly, at least once in two weeks, or three weeks or four weeks or five weeks or a longer time period.

Dose ranges of an AON, composition, compound or adjunct compound according to the invention are preferably designed on the basis of rising dose studies in clinical trials (in vivo use) for which rigorous protocol requirements exist. An AON according to the invention may be used at a dose which is ranged from 0.01 and 20 mg/kg, preferably from 0.05 and 20 mg/kg. In a preferred embodiment, a viral vector, preferably an AAV vector as described earlier herein, as delivery vehicle for an AON according to the invention, is administered in a dose ranging from 1×1⁰⁹-1×10¹⁷ virus particles per injection, more preferably from 1×10¹⁰-1×10¹² virus particles per injection.

The ranges of concentration or dose of AONs as depicted above are preferred concentrations or doses for in vivo, in vitro or ex vivo uses. The skilled person will understand that depending on the AONs used, the target cell to be treated, the gene target and its expression levels, the medium used and the transfection and incubation conditions, the concentration or dose of AONs used may further vary and may need to be optimized any further.

An AON according to the invention, a set of antisense oligonucleotides according to the invention, or a viral vector according to the invention, or a composition according to the invention for use according to the invention may be administered to a cell, tissue and/or an organ in vivo of individuals already affected or at risk of developing a RIPOR2 related disease or a condition requiring degradation of mutated RIPOR2 transcripts, and may be administered in vivo, ex vivo or in vitro. An AON according to the invention, or a viral vector according to the invention, or a composition according to the invention may be directly or indirectly administered to a cell, tissue and/or an organ in vivo of an individual already affected by or at risk of developing a RIPOR2 related disease and may be administered directly or indirectly in vivo, ex vivo or in vitro.

The invention further provides for a method for degrading mutated RIPOR2 transcripts in a cell, said method comprising contacting the cell, preferably a cell expressing mutated RIPOR2, with an antisense oligonucleotide according to the invention, a set of antisense oligonucleotides according to the invention, the vector according to the invention or the pharmaceutical composition according to the invention. The features of this aspect are preferably those defined earlier herein. Contacting the cell with an AON according to the invention, a set of antisense oligonucleotides according to the invention, or a viral vector according to the invention, or a composition according to the invention may be performed by any method known by the person skilled in the art. Use of the methods for delivery of AONs viral vectors and compositions as described earlier herein is included. Contacting may be directly or indirectly and may be in vivo, ex vivo or in vitro.

Unless otherwise indicated each embodiment as described herein may be combined with another embodiment as described herein.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 5% of the value.

The sequence information as provided herein should not be so narrowly construed as to require inclusion of erroneously identified bases. The skilled person is capable of identifying such erroneously identified bases and knows how to correct for such errors.

All patent and literature references cited in the present specification are hereby incorporated by reference in their entirety.

### DESCRIPTION OF THE FIGURES

FIG. 1: Design of RIPOR2-targeting AONs. (A) In silico prediction of the most-probable structure of the mutant RIPOR2 (pre-)mRNA. The 12-nucleotide target deletion breakpoint-nucleotides are marked in red. The mRNA conformation was analyzed using the mfold Web Server, which revealed a mixture of open (non-base paired) and closed nucleotides. (B) Design of 20-mer antisense oligonucleotides (AONs) spanning the 12-nucleotide target deletion (dotted lines). AONs 1-3 consist of a complete phosphorothioate (PS)-linked DNA backbone, whereas AONs 6 and 7 are PS-linked gapmer molecules that contain a DNA gap flanked by 2′-O-methyl RNA wings (2′-OMe, depicted in dark gray).

FIG. 2: Screening of candidate antisense oligonucleotides in patient-derived fibroblasts. Degradation of mutant RIPOR2 transcripts in DFNA21 patient-derived fibroblast cells by antisense oligonucleotides (AONs) (100 nM in the culture medium), directed against the c.1696_1707del RIPOR2 variant. (A) All five phosphorothioate (PS)-modified DNA AONs (1-3) were able to significantly reduce mutant transcript levels, 24 hours after transfection. (B) Gapmer AONs (AON6 and AON7) were designed based on sequences of AON1 and AON2, respectively. Both AONs were able to significantly decrease mutant RIPOR2 transcript levels. Data are expressed as mean±SEM of three replicate transfections, normalized to the expression of GUSB and displayed as the fold change compared to cells treated with transfection reagent only (vehicle). ***p<0.001, ****p<0.0001, one-way ANOVA with Tukey's post-test.

FIG. 3: Dose-response analysis of AON6 in patient-derived fibroblasts. To validate the potency of the previously identified lead molecule, AON6, the efficacy and specificity of the molecule was evaluated at different concentrations (50 nM-250 nM in the medium) in DFNA21 patient-derived fibroblast cells. The scrambled control AON was delivered at 250 nM. A significant decrease in mutant RIPOR2 transcripts 24 hours after transfection was observed at concentrations 150 nM. Data are expressed as mean±SEM of three replicate transfections, normalized to the expression of GUSB and displayed as the fold change compared to cells treated with transfection reagent only (vehicle). **p<0.01, ***p<0.001, one-way ANOVA with Tukey's post-test.

FIG. 4: Evaluation of efficacy of AON6 in HEK293T cells. HEK293T cells were co-transfected with mutant RIPOR2 cDNA constructs and AON6 (250 nM in the medium). RNA and proteins were isolated 24 hours afterwards. (A) RT-qPCR analysis revealed a significant reduction of mutant RIPOR2 transcript cells when HEK293T cells were treated with AON6. Data are expressed as mean±SEM of six replicate transfections, normalized to the expression of GUSB and displayed as the fold change compared to cells transfected with RIPOR2-encoding plasmid and transfection reagent only (plasmid). Vehicle transfected cells were transfected with transfection reagent only and show no endogenous RIPOR2 expression. (B-C) Western blot analyses was performed using anti-RIPOR2 and anti-tubulin antibodies. Analyses confirmed a reduction in mutant RIPOR2 protein synthesis in cells treated with AON6. Quantification of western blot results was performed using the Fiji software (v1.47). Data are expressed as mean±SEM of three replicate transfections, the relative ratio of RIPOR2 protein to tubulin protein was calculated and compared to cells treated with transfection reagent and RIPOR2 only (plasmid). ****p<0.0001, one-way ANOVA with Tukey's post-test.

FIG. 5: Evaluation of efficacy of AON6 on the expression of mutant and wildtype RIPOR2 transcript and protein. HEK293T cells were co-transfected with AONs (150 nM in the medium) and cDNA constructs encoding mutant or wildtype HA-tagged versions of the cochlea-dominant RIPOR2 isoform. RNA and proteins were isolated 24 hours afterwards. (A) RT-qPCR analysis revealed a significant reduction of mutant RIPOR2 transcript cells when HEK293T cells were treated with AON6 compared to scrambled control AON (CTRL AON)-treated samples. The same concentration of AON6 did not result in a decrease in wildtype RIPOR2 transcripts compared to CTRL AON-treated samples. Data are expressed as mean±SEM of three replicate transfections, and normalized to the expression of GUSB. **p<0.01, one-way ANOVA with Tukey's post-test. (B-C) Western blot analyses were performed using anti-RIPOR2 and anti-actin antibodies. B) Synthesis of mutant, HA-tagged RIPOR2 is reduced in cells treated with AON6 as compared to untreated and control AON-treated cells. C) Synthesis of wildtype, HA-tagged RIPOR2 is unaffected by treatment with AON6. Note that native RIPOR2 is also detected by this antibody, and has a slightly lower molecular mass than HA-tagged wildtype RIPOR2.

FIG. 6: Evaluation of the effects of AON6 with and without 8-oxoG modification at position 16 on mutant RIPOR2 transcript levels. HEK293T cells were co-transfected with cDNA constructs encoding HA-tagged versions of mutant, cochlea-dominant RIPOR2 isoform, and AON6 with and without 8-oxoG modification, the scrambled control AON (CTRL AON). RNA was isolated 24 hours after transfection, and RIPOR2 transcript levels were assessed by RT-qPCR. A significant reduction of mutant RIPOR2 transcripts was observed after transfection with AON6 and 8-oxoG AON6. At both 100 nM and 250 nM, no significant differences were observed between the reduction in mutant RIPOR2 transcripts induced by AON6 and 8-oxoG AON. Data are expressed as the mean±SEM of three replicate transfections, normalized to the expression of GUSB. Untransfected cells show no endogenous RIPOR2 expression. **** p<0.0001 vs CTRL AON treated cells, one-way ANOVA with Tukey's post-test.

### DESCRIPTION OF THE SEQUENCES

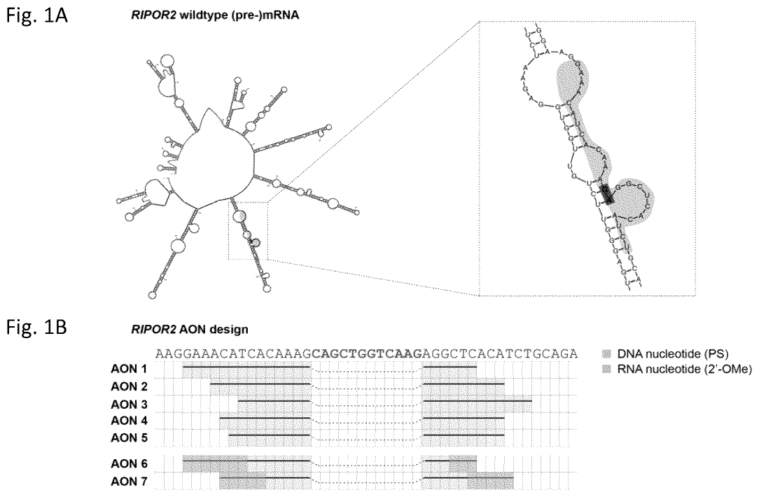

TABLE 1
Sequences

| SEQ ID | |
|---|---|
| NO: | Name |
| 1 | mRNA RIPOR2 c.1696_1707del |
| 2 | target c.1696_1707del + 10 nt |
| 3 | target c.1696_1707del + 5 nt |
| 4 | target c.1696_1707del |
| 5 | AON1 |
| 6 | AON2 |
| 7 | AON3 |
| 8 | AON4 |
| 9 | AON5 |
| 10 | AON6 |
| 11 | AON7 |
| 12 | cDNA sequences encoding an N-terminally FLAG-tagged |
| | mutant RIPOR2 |
| 13 | mutant RIPOR2-specific forward primer |
| 14 | universal RIPOR2 reverse primer |
| 15 | GUSB forward primer |
| 16 | GUSB reverse primer |
| 17 | Scrambled control AON |
| 18 | Sequence FIG. 1 |
| 19 | cDNA sequences encoding a C-terminally HA-tagged mutant |
| | RIPOR2 (cochlea dominant isoform) |
| 20 | cDNA sequences encoding a C-terminally HA-tagged wildtype |
| | RIPOR2 (cochlea dominant isoform) |
| 21 | 8-oxOG AON6 |

### EXAMPLES

### Materials and Methods

### Design of Antisense Oligonucleotides

AONs were designed following previously described criteria.[13-15] Exon 14, harboring the 12-nucleotide (nt) target deletion, was examined for open configuration (mfold Web Server [16]). Thermodynamic properties of potential 20-mer AON molecules (40-60% GC content) were assessed using the RNAstructure software as previously described.[15] Uniqueness of AON target sequences was validated using BLAST (NCBI), allowing a maximum of two mismatches. AONs were purchased from Eurogentec and dissolved in PBS at a concentration of 1 mM. Sequences and AON chemistry are provided in Table 2.

TABLE 2
AONs tested

| AON | SEQ ID NO | Sequence |
|---|---|---|
| AON1 | 5 | G*A*G*C*C*T*C*T*T*T*G*T*G*A*T*G*T*T*T*C |
| AON2 | 6 | T*G*T*G*A*G*C*C*T*C*T*T*T*G*T*G*A*T*G*T |
| AON3 | 7 | A*G*A*T*G*T*G*A*G*C*C*T*C*T*T*T*G*T*G*A |
| AON4 | 8 | T*G*T*G*A*G*C*C*T*C*T*T*T*G*T*G*A*T*G |
| AON5 | 9 | T*G*T*G*A*G*C*C*T*C*T*T*T*G*T*G*A*T |
| AON6 | 10 | (G*A*G*)C*C*T*C*T*T*T*G*T*G*(A*U*G*U*U*U*C) |
| AON7 | 11 | (A*U*G*U*G*)A*G*C*C*T*C*T*T*T*G*(U*G*A*U*G) |
| 8-oxo | 21 | (G*A*G*)C*C*T*C*T*T*T*G*T*G*(A*U*8oxoG*U*U*U*C) |
| AON6 | | |

Phosphorothioate links in the antisense oligonucleotide (AON) sequences are indicated by the asterisks between bases. The 2′-O-methyl RNA bases are placed between brackets. The nucleotides directly flanking the c.1696_1707del variant in RIPOR2 are indicated by bold underlined fonts.

### Cell Culture Conditions and AON Delivery

Patient-derived primary fibroblast cells were cultured in standard fibroblast medium consisting of DMEM (Gibco) supplemented with 20% fetal calf serum, 1% sodium pyruvate and 1% penicillin-streptomycin. Prior to AON treatment, cells were seeded in 12 wells plates and cultured to a confluency of ^{˜}80%. Cells were transfected with AON molecules (final concentrations 50-250 nM in the culture medium) using Lipofectamine 2000 (Invitrogen) according to the manufacturer's instructions, following a 1:2 ratio (1 μg AON:2 μl Lipofectamine reagent). After 24 hours, cells were harvested for RNA isolation and subsequent transcript analysis.

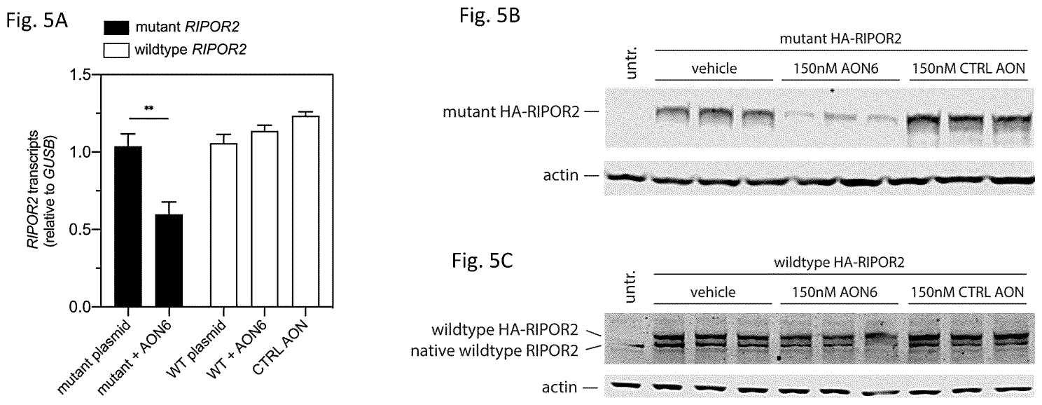

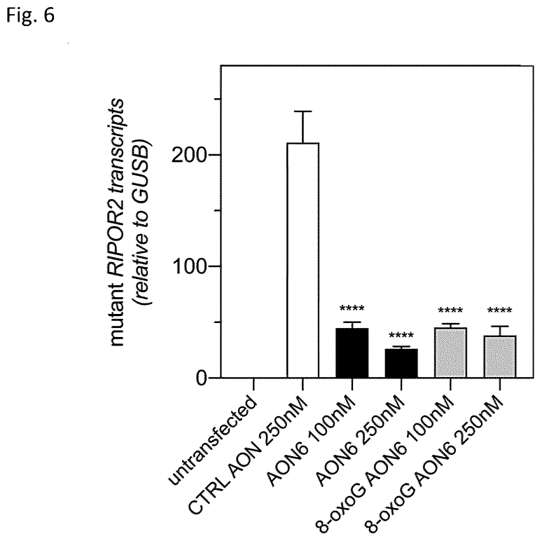

For AON delivery in HEK293T cells, plasmids containing cDNA sequences encoding an N-terminally FLAG-tagged mutant RIPOR2 (NM_015864.3; SEQ ID NO: 12) were generated with Gateway Technology (Life Technologies). RNA isolated from patient-derived EBV-transformed lymphoblastoid cells was used as input for Gateway-adapted RT-PCR. The sequence of the clones was verified with Sanger sequencing. HEK293T cells were co-transfected with AONs and the generated DNA constructs (500 ng per well) using 45 μl polyethyleneimine (PEI) [17]. Treated cells were collected 24 hours after AON delivery for transcript and protein analyses. For the experiments shown in FIGS. 5 and 6, C-terminally HA-tagged mutant and wildtype protein-coding sequences of the longer, cochlea dominant RIPOR2 isoform were used (NM_001346031.1; SEQ ID NO: 19 (mutant) and SEQ ID NO: 20 ((wildtype)). Both isoform NM_015864.3 and NM_001346031.1 contain the AON target sequence.

### RNA Isolation and RT-qPCR

Total RNA was isolated from treated cells using the Nucleospin RNA kit (Machery-Nagel) according to the manufacturer's instructions. Subsequently, cDNA was synthesized using the iScript cDNA synthesis kit (Bio-Rad) and 250 ng RNA as input material. Obtained cDNA was diluted twice (fibroblast cells) or five times (HEK293T cells) for quantitative PCR analysis.

QPCR analysis was performed using GoTaq qPCR Master Mix (Promega) according to standard procedures. Allele-specific primer sets were employed that allowed to distinguish the mutant RIPOR2 transcripts and included mutant RIPOR2-specific forward primer (5′-GGAAGGAAACATCACAAAGAG-3′; SEQ ID NO: 13, and a universal RIPOR2 reverse primer (5′-GCAGCCTTCAGATTCTCC-3′; SEQ ID NO: 14). Relative expression levels as compared to the housekeeping gene GUSB (primers 5′-AGAGTGGTGCTGAGGATTGG-3′; SEQ ID NO: 15, and 5′-CCCTCATGCTCTAGCGTGTC-3′; SEQ ID NO: 16) were determined with the 2\hat{ }ΔCt method.

### Western Blot

For protein isolation, cells were treated with a lysis buffer containing 50 mM Tris pH 7.5, 150 mM NaCl and 0.5% (v/v) Triton X-100. Protein lysates were supplemented with protein sample loading buffer (LI-COR) and DTT (final concentration 2 mM) and denatured at 70° C. for 10 minutes. Proteins were separated on a 4-12% NuPAGE Bis-Tris gel (ThermoFisher) in NuPAGE MOPS SDS running buffer (ThermoFisher) and transferred overnight at 4° C. in NuPAGE transfer buffer supplemented with 20% methanol to a nitrocellulose membrane (0.45 μm, Sigma-Aldrich). Membranes were blocked with 5% Blotto, non-fat dry milk, blocking buffer (Santa Cruz), and incubated with primary antibodies (1 hour, RT) and secondary antibodies (45 minutes, RT), in 0.5% blocking buffer. In between incubation steps, the membranes were washed three times for 10 minutes with PBS supplemented with 0.2% Tween-20. After a final wash with PBS, proteins were visualized using the Odyssey Infrared Imaging System (LI-COR). RIPOR2 protein was quantified using the Fiji software (version 1.47), and normalized against tubulin. Antibodies: anti-RIPOR2 (1:1000; cat. #17015-1-AP, Proteintech), anti-tubulin (1:2000; cat. #ab7291, Abcam), goat anti-rabbit Alexa Fluor 680 (1:20000; cat. #A21076, Molecular Probes) and goat anti-mouse IRDye800 (1:20000; cat. #926-32210, LI-COR). In FIG. 5, actin was visualized as loading control using anti-actin antibodies (#MA5-11869, Thermo Fischer Scientific) that recognize all vertebrate non-muscular actins.

### Results

### Design of Allele-Specific RNase H1-Dependent AONs

We first employed several in silico analyses in order to design RNase H1-dependent AONs complementary to transcripts of the c.1696_1707del mutant, but not wildtype, RIPOR2 allele. First, internal hybridization of the (pre-)mRNA of mutant RIPOR2 exon 14 was predicted in silico to determine accessibility of the target region (FIG. 1A). In the putative AON target region, 17 of the 30 nucleotides are unpaired in the most probable 3D structure. Several candidate AONs were selected that span the 12nt-deletion region, which is a prerequisite for allele-discrimination. Next, thermodynamic properties were calculated, among which the free energy of on- and off-target AON binding, and the potential of the AONs for hairpin formation and dimerization. Based on these in silico predictions, three AON molecules were ordered as phosphorothioate (PS)-linked DNA-bases for an initial screening. FIG. 1B provides a schematic overview of the target regions of AONs that are used in this study.

### AONs Induce Degradation of Mutant RIPOR2 Transcripts in Patient-Derived Fibroblast Cells

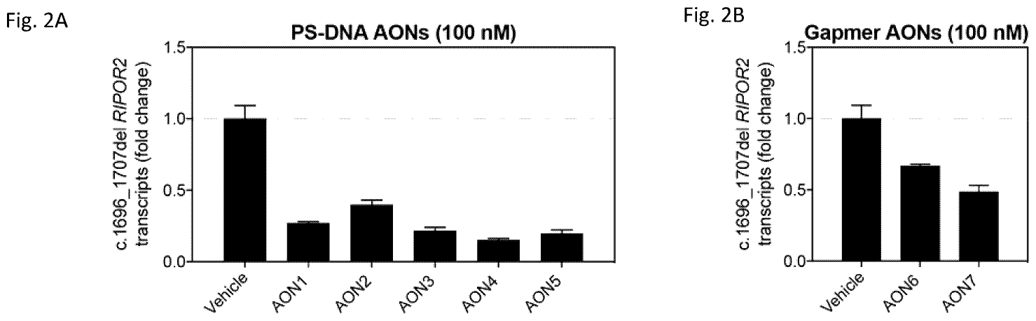

To assess the potency of the AON strategy and the accessibility of the AON-target region of the mutant RIPOR2 transcript, DFNA21 patient-derived primary fibroblast cells were treated with PS-DNA oligos at a final concentration of 100 nM in the culture medium. This dose was selected based on earlier findings described by de Vrieze et al.[11] where significant reduction in mutant COCH transcripts was achieved with similarly designed AONs. The non-gapmer composition was selected for its strong ability to recruit RNase H1. All five c.1696_1707del-targeting AON molecules were able to induce a significant reduction in transcript levels of the mutant allele (ranging from 60-85%) compared to cells that were treated with transfection reagent only, indicating that the target region in mutant RIPOR2 is indeed accessible to AONs (FIG. 2A).

As a next step, AON1 (SEQ ID NO: 5) and AON2 (SEQ ID NO: 6) were ordered with a gapmer chemistry that included a central gap region of PS-DNA bases flanked by 2′-O-methyl RNA wings. Although the gapmer composition is known to decrease RNase H1 cleavage efficiency, their improved stability and reduced toxicity, make them particularly attractive for clinical applications. In addition, the inability of RNase H1 to cleave 2′-O-methyl RNA bases was exploited to improve allele-specificity. In view of the preferred cleavage preference of RNase H1 [18], an asymmetric wing design was preferred for the gapmer of AON1. This resulted in AON6 (SEQ ID NO: 10), which is comprised of a 7-10-3 design. Symmetric wings were chosen for the gapmer of AON2 (FIG. 1). Additionally, the resulting AON7 (SEQ ID NO: 11) is also shifted by one nucleotide compared to AON2. Treatment of cells with AON6 and AON7 also both led to significant reduction of mutant RIPOR2 transcript levels (33% and 51%, respectively) as compared to treatment with transfection reagent only (FIG. 2B). The decrease in mutant RIPOR2 transcripts appears lower compared to that achieved by treatment with PS-DNA AONs. However, the PS-DNA AONs are less suitable for clinical use due to their relatively poor nuclease resistance and sequence-specific hybridization properties as compared to gapmer AONs [19].

AON6 Induces a Dose-Dependent and Specific Decrease in Mutant RIPOR2 transcript Levels

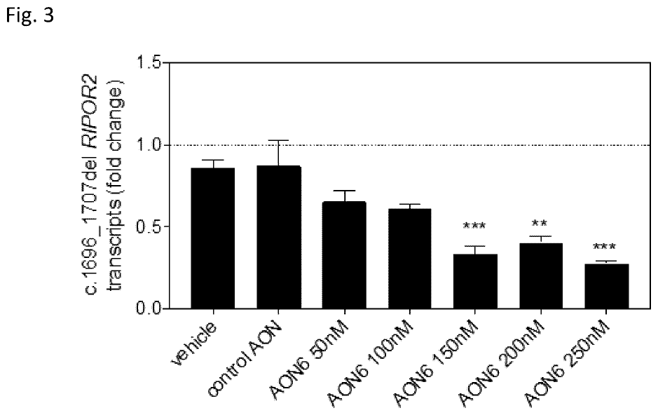

To further investigate the efficacy and allele-specificity of AON6, a dose-response analysis was performed. The AONs were transfected in patient-derived fibroblast cells, with a final concentration in the culture medium ranging from 50 nM to 250 nM. A significant reduction in transcripts of the mutant allele could be observed when treated with concentrations ≥150 nM (FIG. 3). A maximum knockdown of 68% was achieved as compared to vehicle-treated and scrambled control AON (SEQ ID NO: 17) treated cells. The observation that AON6, but not the control AON, reduces mutant RIPOR2 transcript levels indicates AON6 is able to target the mutant RIPOR2 allele for RNase H1-mediated degradation in a sequence-specific manner.

### Validation of the Lead AON Molecule in HEK293T Cells

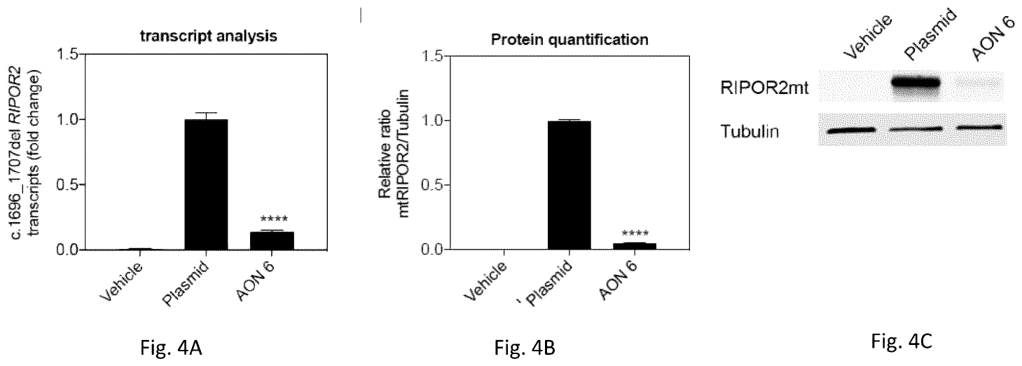

We furthermore questioned whether AON6 could decrease the production of the mutant RIPOR2 protein. We co-transfected HEK293T cells with DNA constructs encoding FLAG-tagged mutant RIPOR2 and AON6. The maximum AON concentration (250 nM) that was tested in fibroblast cells was selected for this experiment. Again, a strong decrease of mutant RIPOR2 transcripts was observed (88% reduction; p-value <0.0001, ****) (FIG. 4A). Additionally, we performed a western blot analysis to confirm the effect of AON6 on mutant RIPOR2 translation (FIG. 4B, C). In three replicate AON deliveries, western blot analysis of flag-tagged mutant RIPOR2 revelated a strong decrease in mutant RIPOR2 translation (95% reduction; p-value <0.0001, ****). This indicates that the AON molecule is also able to effectively lower the amount of mutant RIPOR2 protein production.

### AON6 Specifically Reduces Mutant RIPOR2 Protein Levels

Multiple transcripts encoding different isoforms of the RIPOR2 protein have been described. In previous experiments, a plasmid encoding the short isoform (SEQ ID NO: 12) was used to drive expression of mutant RIPOR2 in HEK293T cells. In the subsequent experiment, we transfected HEK293T cells with plasmids expression the longer, cochlea-dominant, mutant and wildtype RIPOR2 transcripts (SEQ ID NO:19 and SEQ ID NO: 20, respectively) to assess the effect of AON6 on RIPOR2 protein expression. Both isoforms contain the DFNA21-associated 12nt deletion, and are targets for AON-induced transcript degradation.

First, we assessed the effect of AON6 on mutant and wildtype transcript levels encoding this longer RIPOR2 isoform. Based on the results obtained with AON6 from experiments in patient-derived fibroblasts (FIG. 3), we chose to deliver AON6 at a final concentration in the culture medium of 150 nM in the current experiment. At this dose, AON6 was able to significantly reduce mutant RIPOR2 transcripts (40% reduction; p-value <0.01, *, one-way ANOVA with Tukey multiple comparison test), but had no effect on wildtype RIPOR2 transcript levels (FIG. 5A). Next, we assessed the effect of AON6 treatment on RIPOR2 translation. Protein lysates from AON6—and scrambled control AON (CTRL AON; SEQ ID NO: 17)-treated cells were investigated using SDS-Page gel electrophoresis and Western Blotting with RIPOR2-specific antibodies. As evident by the small and less intense band corresponding to mutant RIPOR2 in cells treated with 150 nM AON6 as compared to controls, AON6 is able to induce a near complete inhibition of mutant RIPOR2 protein synthesis (FIG. 5B). In wildtype RIPOR2-transfected cells, co-delivery of AON6 had no effect on wildtype RIPOR2 protein levels (FIG. 5C). The observed effects on protein level are larger as compared to those observed on transcript level. This suggest that, besides RNase H1-mediated breakdown of mutant transcripts, AONs could sterically hinder the translation machinery on mutant RIPOR2 transcripts that are not (yet) cleaved by RNase H1.

### Chemical Modification of AON6

The nucleotides of an AON can be chemically modified to introduce favorable properties in the AON, and modulate amongst others the AON affinity for the RNA target. One example is the 8-oxo-guanine (8-oxoG) modification, which is an oxidized version of a regular guanine (G) that has a reduced affinity for cytosine (C), but can form base-pairs with adenine (A). By introducing the 8-oxoG in an AON sequence, the on-target binding affinity can be slightly reduced, which can be advantageous to improve allele-specificity. Here, we introduced an 8-oxoG nucleotide on position 16 of AON6 (8-oxoG AON6, SEQ ID NO: 21) to assess if on-target affinity of AON6 can be lowered without affecting the molecular efficacy on transcript level. HEK293T cells were transfected with mutant RIPOR2 constructs (cochlea-dominant isoform) and regular AON6, 8-oxoG AON6 or control AON. At a concentration of 100 nM, both AON6 and 8-oxoG AON6 resulted in a significant decrease in mutant RIPOR2 transcript levels (both 79% reduction vs CTRL AON; p >0.0001)(FIG. 6). At 250 nM, the effect of both AONs on mutant RIPOR2 transcript level is slightly larger compared to the effect observed at 100 nM, but this increased reduction is not statistically significant (AON6: p=0.85; 8-oxoG AON6: p=0.99, one-way ANOVA with Tukey multiple comparison test). This result indicates that chemical modifications can be introduced in AON6 without affecting the ability of this AON to reduce mutant RIPOR2 transcript levels.

### CONCLUSION

Overall, our results demonstrate that the delivery of antisense oligonucleotides can be used to decrease the levels of mutant RIPOR2 transcripts and mutant RIPOR2 protein synthesis. DFNA21 is a dominantly inherited disease, where the protein encoded by the mutant RIPOR2 gene interferes with normal function of the cochlea. DFNA21 patients all have a single healthy copy of the RIPOR2 gene that, in absence of mutant RIPOR2 proteins, is sufficient to produce levels of wildtype RIPOR2 high enough for normal function of the inner ear [4,6]. Therefore, AONs according to the invention can be used in the treatment of human subjects suffering from hearing impairment type DFNA21 resulting from dominantly-inherited mutations in the RIPOR2 gene.

### REFERENCES

(1) Bruijn, S. E. de; Smits, J. J.; Liu, C.; Lanting, C. P.; Beynon, A. J.; Blankevoort, J.; Oostrik, J.; Koole, W.; Vrieze, E. de; Cremers, C.; Cremers, F. P. M.; Roosing, S.; Yntema, H. G.; Kunst, H. P. M.; Zhao, B.; Pennings, R. J. E.; Kremer, H. A RIPOR2 In-Frame Deletion Is a Frequent and Highly Penetrant Cause of Adult-Onset Hearing Loss. Journal of Medical Genetics 2021, 58, 96-104. https://doi.org/10.1136/jmedgenet-2020-106863.
(2) Kunst, H.; Marres, H.; Huygen, P.; Duijnhoven, G. V.; Krebsova, A.; Velde, S. V. der; Reis, A.; Cremers, F. P. M.; Cremers, C. Non-Syndromic Autosomal Dominant Progressive Non-Specific Mid-Frequency Sensorineural Hearing Impairment with Childhood to Late Adolescence Onset (DFNA21). Clinical Otolaryngology & Allied Sciences 2000, 25, 45-54. https://doi.org/doi:10.1046/j.1365-2273.2000.00327.x.
(3) Brouwer, A. P. M. D.; Kunst, H. P. M.; Krebsova, A.; Asseldonk, K. van; Reis, A.; Snoeckx, R. L.; Camp, G. V.; Cremers, C. W. R. J.; Cremers, F. P. M.; Kremer, H. Fine Mapping of Autosomal Dominant Nonsyndromic Hearing Impairment DFNA21 to Chromosome 6p 24.1-22.3. American Journal of Medical Genetics 2005, 137A, 41-46. https://doi.org/doi:10.1002/ajmg.a.30844.
(4) Zhao, B.; Wu, Z.; Müller, U. Murine Fam65b Forms Ring-like Structures at the Base of Stereocilia Critical for Mechanosensory Hair Cell Function. eLife 2016, 5, e14222. https://doi.org/10.7554/elife.14222.
(5) Diaz-Horta, O.; Abad, C.; Cengiz, F. B.; Bademci, G.; Blackwelder, P.; Walz, K.; Tekin, M. Ripor2 Is Involved in Auditory Hair Cell Stereociliary Bundle Structure and Orientation. Journal of Molecular Medicine 2018, 96 (11), 1227-1238. https://doi.org/10.1007/s00109-018-1694-x.
(6) Diaz-Horta, O.; Subasioglu-Uzak, A.; Grati, M.; DeSmidt, A.; Foster, J.; Cao, L.; Bademci, G.; Tokgoz-Yilmaz, S.; Duman, D.; Cengiz, F. B.; Abad, C.; Mittal, R.; Blanton, S.; Liu, X. Z.; Farooq, A.; Walz, K.; Lu, Z.; Tekin, M. FAM65B Is a Membrane-Associated Protein of Hair Cell Stereocilia Required for Hearing. Proceedings of the National Academy of Sciences 2014, 111 (27), 9864-9868. https://doi.org/10.1073/pnas.1401950111.
(7) Vickers, T. A.; Crooke, S. T. The Rates of the Major Steps in the Molecular Mechanism of RNase H1-Dependent Antisense Oligonucleotide Induced Degradation of RNA. Nucleic acids research 2015, 43 (18), 8955-8963. https://doi.org/10.1093/nar/gkv920.
(8) Crooke, S. T. Molecular Mechanisms of Action of Antisense Drugs. Biochimica et Biophysica Acta 1999, 1489 (1), 31-44. https://doi.org/10.1016/s0167-4781(99)00148-7.
(9) Vickers, T. A.; Crooke, S. T. Antisense Oligonucleotides Capable of Promoting Specific Target MRNA Reduction via Competing RNase H1-Dependent and Independent Mechanisms. PLoS One 2014, 9 (10), e108625. https://doi.org/10.1371/journal.pone.0108625.
(10) Crooke, S. T.; Baker, B. F.; Crooke, R. M.; Liang, X. Antisense Technology: An Overview and Prospectus. Nat Rev Drug Discov 2021, Online ahead of print, 1-27. https://doi.org/10.1038/s41573-021-00162-z.
(11) Vrieze, E. de; Martín, J. C.; Peijnenborg, J.; Martens, A.; Oostrik, J.; Heuvel, S. van den; Neveling, K.; Pennings, R.; Kremer, H.; Wijk, E. van. Antisense Oligonucleotide-Based Degradation of c.151C>T Mutant COCH Transcripts Associated with Dominantly Inherited Hearing Impairment DFNA9. Mol Ther—Nucleic Acids 2021, 24, 274-283. https://doi.org/10.1016/j.omtn.2021.02.033.
(12) Hammond, S. M.; Aartsma-Rus, A.; Alves, S.; Borgos, S. E.; Buijsen, R. A. M.; Collin, R. W. J.; Covello, G.; Denti, M. A.; Desviat, L. R.; Echevarría, L.; Foged, C.; Gaina, G.; Garanto, A.; Goyenvalle, A. T.; Guzowska, M.; Holodnuka, I.; Jones, D. R.; Krause, S.; Lehto, T.; Montolio, M.; Roon-Mom, W. V.; Arechavala-Gomeza, V. Delivery of Oligonucleotide-Based Therapeutics: Challenges and Opportunities. EMBO Molecular Medicine 2021, 13, e13243. https://doi.org/https://doi.org/10.15252/emmm.202013243.
(13) Pallan, P. S.; Egli, M. Insights into RNA/DNA Hybrid Recognition and Processing by RNase H from the Crystal Structure of a Non-Specific Enzyme-DsDNA Complex. Cell Cycle 2008, 7 (16), 2562-2569. https://doi.org/10.4161/cc.7.16.6461.
(14) Aartsma-Rus, A.; Vliet, L. van; Hirschi, M.; Janson, A. A. M.; Heemskerk, H.; Winter, C. L. de; Kimpe, S. de; Deutekom, J. C. T. van; Hoen, P. A. C. t; Ommen, G.-J. B. van. Guidelines for Antisense Oligonucleotide Design and Insight into Splice-Modulating Mechanisms. Molecular Therapy 2009, 17 (3), 548-553. https://doi.org/10.1038/mt.2008.205.
(15) Slijkerman, R.; Kremer, H.; Wijk, E. van. Antisense Oligonucleotide Design and Evaluation of Splice-Modulating Properties Using Cell-Based Assays. Methods in Molecular Biology 2018, 1828 (2), 519-530. https://doi.org/10.1007/978-1-4939-8651-4_34.
(16) Zuker, M. Mfold Web Server for Nucleic Acid Folding and Hybridization Prediction. Nucleic acids research 2003, 31, 3406-3415. https://doi.org/10.1093/nar/gkg595.
(17) Roosing, S.; Lamers, I. J.; Vrieze, E. de; Born, L. I. van den; Lambertus, S.; Arts, H. H.; Peters, T. A.; Hoyng, C. B.; Kremer, H.; Hetterschijt, L.; Letteboer, S. J.; Wijk, E. van; Roepman, R.; Hollander, A. I. den; Cremers, F. P. Disruption of the Basal Body Protein POC1B Results in Autosomal-Recessive Cone-Rod Dystrophy. American Journal of Medical Genetics 2014, 95 (2), 131-142. https://doi.org/10.1016/j.ajhg.2014.06.012.
(18) Kielpiński, Ł. J.; Hagedorn, P. H.; Lindow, M.; Vinther, J. RNase H Sequence Preferences Influence Antisense Oligonucleotide Efficiency. Nucleic acids research 2017, 45 (22), 12932-12944. https://doi.org/10.1093/nar/gkx1073.
(19) Smith, C. I. E.; Zain, R. Therapeutic Oligonucleotides: State of the Art. Annual review of pharmacology and toxicology 2019, 59 (1), 605-630. https://doi.org/10.1146/annurev-pharmtox-010818-021050.

1. An antisense oligonucleotide moiety for the degradation of a mutated RIPOR2 transcript that binds to and/or is complementary to a polynucleotide with the nucleotide sequence as set forward in SEQ ID NO: 1.

2. An antisense oligonucleotide for the degradation of a mutated RIPOR2 according to claim 1, wherein the antisense oligonucleotide comprises an RNA residue, a DNA residue, and/or a nucleotide analogue or equivalent.

3. An antisense oligonucleotide for the degradation of a mutated RIPOR2 according to claim 1, wherein the antisense nucleotide is a gapmer.

4. An antisense oligonucleotide for the degradation of a mutated RIPOR2 according to claim 1, wherein the antisense oligonucleotide has a length of from about 8 to about 40 nucleotides.

5. An antisense oligonucleotide for the degradation of a mutated RIPOR2 according to a claim 1, wherein said antisense oligonucleotide comprises or consists of an oligonucleotide with the sequence as set forward in SEQ ID NO: 5, 6, 7, 8, 9, 10 11 and 21.

6. An antisense oligonucleotide for the degradation of a mutated RIPOR2 according to claim 1, comprising a 2′-O alkyl phosphorothioate modified nucleotide, such as a 2′-O-methyl modified ribose, a 2′-O-ethyl modified ribose, a 2′-O-propyl modified ribose, 2-O′-methoxy ethyl-ribose and/or substituted derivatives of these modifications such as halogenated derivative.

7. A pharmaceutical composition comprising an antisense oligonucleotide for the degradation of a mutated RIPOR2 according to claim 1 and further comprising a pharmaceutically acceptable excipient.

8. A pharmaceutical composition according to claim 7, wherein the pharmaceutical composition is for administration into the cochlea.

9.-10. (canceled)

11. A method of treatment of a RIPOR2 related disease or condition requiring the degradation of mutated RIPOR2 (pre)mRNA in a subject in need thereof, comprising administration of an antisense oligonucleotide for the degradation of a mutated RIPOR2 as defined in claim 1.

12. (canceled)

13. The method according to claim 11, wherein the RIPOR2 related disease or condition is a condition resulting in hearing impairment and/or vestibular dysfunction.

14. The antisense oligonucleotide for the degradation of a mutated RIPOR2 according to claim 1, wherein the antisense oligonucleotide moiety binds to or is complementary to a polynucleotide part within SEQ ID NO: 1.

15. The antisense oligonucleotide for the degradation of a mutated RIPOR2 according to claim 14, wherein the polynucleotide part has a nucleotide sequence selected from the group consisting of SEQ ID NO: 2, 3, and 4.

16. The antisense oligonucleotide for the degradation of a mutated RIPOR2 according to claim 6, wherein the antisense oligonucleotides comprise an 8-oxoGuanine (8-oxoG) modification.

17. The method according to claim 13, wherein the condition is a vestibulo-cochlear disorder.

18. The method according to claim 13, wherein the disease or condition is DFNA21.


SEQUENCE LISTING (21 sequences)

SEQ ID NO: 1 - DNA, 3195 bp, Homo sapiens
atgttggtaggatcccagtctttttcgcctggagggcccaatgggatcattagaagccagtcctttgcgggtttcagcggcctccaggaaaggcgatccaggtgtaactccttcattgaaaattcctccgctctcaagaagcctcaggccaaactgaagaaaatgcacaatttaggccacaaaaacaacaatccccccaaagagcctcagcctaaaagggtggaagaagtctacagggccttgaaaaatggacttgatgaatatctggaggttcaccagacggagctggacaagttgacagctcagttaaaagatatgaaaagaaactctcgcctgggtgtactgtatgacctagacaagcaaattaaaacaattgaaagatacatgagacgcctggagtttcatataagtaaggtagatgaactctatgaagcttattgtatccagcgacgcctccaggatggtgccagcaaaatgaagcaagccttcgcaacatcccctgccagcaaagctgcccgggagagtctgacagagatcaatcggagcttcaaggagtacacagagaatatgtgcaccattgaagtggagctagagaatctgctgggagaattctccatcaagatgaaaggtctggctggctttgcacgcctctgtcctggagatcaatatgaaattttcatgaagtatggccggcagcggtggaaactgaaaggcaaaatagaagtaaatggcaagcagagctgggatggagaagaaacagtttttctgcccctgatagttgggttcatctccatcaaggtcacggagctcaaagggctagcaactcacatcctggtaggtagcgtgacctgtgagaccaaagagctgtttgcagcccgacctcaggtagtggctgtcgacatcaatgaccttggtaccatcaaactgaacctggaaatcacctggtatccatttgacgtggaggacatgaccgcatcctcaggcgctgggaacaaggcagcagcccttcagaggagaatgtccatgtacagccagggtaccccggaaacgcccaccttcaaagaccactccttctttaggtggctgcatccttccccagacaagcccaggcggctgtctgtcttgagtgccttgcaagacactttctttgccaagctgcaccgcagccgctccttcagtgacctgccctccctcaggccgagtcccaaggccgtgctagagctctattcaaatctacctgatgacatctttgaaaatggaaaggcagccgaggagaaaatgccactgtcgctcagcttcagtgacctgcccaacggggactgcgccctcacctcccactcaacaggctccccttccaactcaacaaatccagaaattaccatcacccctgcggagtttaacctcagcagcttggcctcccagaatgagggtatggatgacaccagctcagcatcttccaggaactccctgggagaaggccaagagccaaagtcacacctgaaggaggaagacccagaggagcccagaaaacctgcctcggccccatctgaggcttgccgccgacagtcctcaggtgctggggctgagcacctgttccttgagaatgatgttgcagaagcacttctgcaagagtctgaggaggcctctgagctcaagcctgtggaactggacacttcggaaggaaacatcacaaagaggctcacatctgcagaggtgccaatggccacagacaggctgctctctgagggttctgttggtggagaatctgaaggctgcagatcctttctagatggaagcttagaggatgcttttaatgggcttttacttgcattagaaccacataaagagcagtataaagagtttcaggatctgaaccaagaagtcatgaatttggatgatattctaaaatgcaagccagcagtaagccgcagcaggtcttccagtttaagtctcacagttgaaagtgctttagaaagctttgatttcctgaacacctctgattttgacgaggaggaggatggtgatgaggtttgtaatgttggcggaggtgctgactcagtattttcagacactgagactgagaaacacagttacaggtcggttcacccagaagccagggggcatctcagtgaagcgctcactgaagacacaggagttgggaccagtgtggcaggaagtcctctcccactgaccacaggcaacgagagcctggacatcaccatcgtcaggcacctccagtactgcacccaactcgtgcagcaaattgttttctcaagcaaaaccccatttgtggcaagaagtctcttagagaagctttctaggcagatccaagtgatggagaaactcgcagctgtcagtgatgagaacataggaaatatcagttctgttgtggaagccataccagaatttcacaaaaagctgtctttgctgtcattctggaccaagtgctgcagccctgttggtgtctaccacagcccagcggacagagtgatgaagcagctggaggccagctttgccagaactgtcaacaaagaatatccaggacttgcagacccagtgtttcgaaccctggtgtcccaaattctggaccgggctgagcctctgctttcctccagcctgtcctcggaagtcgtcactgttttccagtattacagttacttcaccagccacggcgtcagtgacctggagagttacctgagccagctggccaggcaagtttccatggttcagactctgcaatcactaagagatgaaaaactgctacaaaccatgagtgaccttgctcccagcaacctcctggcccagcaggaagtactcaggactctggctctgctattaaccagagaggacaacgaagttagcgaggctgtgacgctttacttggcagcagcctccaaaaatcagcatttcagggaaaaggccttgctctattactgtgaagcactaacaaagacaaacctccagctccagaaagcagcttgcctggctctgaaaatccttgaggctactgaaagcattaaaatgctggtgacattgtgtcaatctgatactgaagaaatcagaaatgtggcctcagaaaccctcttgtctctgggagaagatgggcggctggcatatgaacaattggacaaatttcctcgagactgtgttaaagtcggaggtcgtcatggaactgaagttgccacagccttttaa

SEQ ID NO: 2 - DNA, 60 bp, Homo sapiens
gaactggacacttcggaaggaaacatcacaaagaggctcacatctgcagaggtgccaatg

SEQ ID NO: 3 - DNA, 50 bp, Homo sapiens
ggacacttcggaaggaaacatcacaaagaggctcacatctgcagaggtgc

SEQ ID NO: 4 - DNA, 40 bp, Homo sapiens
cttcggaaggaaacatcacaaagaggctcacatctgcaga

SEQ ID NO: 5 - DNA, 20 bp, Artificial Sequence
  AON-1
gagcctctttgtgatgtttc

SEQ ID NO: 6 - DNA, 20 bp, Artificial Sequence
  AON-2
tgtgagcctctttgtgatgt

SEQ ID NO: 7 - DNA, 20 bp, Artificial Sequence
  AON-3
agatgtgagcctctttgtga

SEQ ID NO: 8 - DNA, 19 bp, Artificial Sequence
  AON-4
tgtgagcctctttgtgatg

SEQ ID NO: 9 - DNA, 18 bp, Artificial Sequence
  AON-5
tgtgagcctctttgtgat

SEQ ID NO: 10 - DNA, 20 bp, Artificial Sequence
  AON6
gagcctctttgtgauguuuc

SEQ ID NO: 11 - DNA, 20 bp, Artificial Sequence
  AON-7
augugagcctctttgugaug

SEQ ID NO: 12 - DNA, 1830 bp, Artificial Sequence
  cDNA sequence encoding an N-terminally FLAG-tagged mutant RIPOR2
atggactacaaagacgatgacgacaagcttgcggccatcacaagtttgtacaaaaaagcaggcttcatgttggtaggatcccagtctttttcgcctggagggcccaatgggatcattagaagccagtcctttgcgggtttcagcggcctccaggaaaggcgatccaggtgtaactccttcattgaaaattcctccgctctcaagaagcctcaggccaaactgaagaaaatgcacaatttaggccacaaaaacaacaatccccccaaagagcctcagcctaaaagggtggaagaagtctacagggccttgaaaaatggacttgatgaatatctggaggttcaccagacggagctggacaagttgacagctcagttaaaagatatgaaaagaaactctcgcctgggtgtactgtatgacctagacaagcaaattaaaacaattgaaagatacatgagacgcctggagtttcatataagtaaggtagatgaactctatgaagcttattgtatccagcgacgcctccaggatggtgccagcaaaatgaagcaagccttcgcaacatcccctgccagcaaagctgcccgggagagtctgacagagatcaatcggagcttcaaggagtacacagagaatatgtgcaccattgaagtggagctagagaatctgctgggagaattctccatcaagatgaaaggtctggctggctttgcacgcctctgtcctggagatcaatatgaaattttcatgaagtatggccggcagcggtggaaactgaaaggcaaaatagaagtaaatggcaagcagagctgggatggagaagaaacagtttttctgcccctgatagttgggttcatctccatcaaggtcacggagctcaaagggctagcaactcacatcctggtaggtagcgtgacctgtgagaccaaagagctgtttgcagcccgacctcaggtagtggctgtcgacatcaatgaccttggtaccatcaaactgaacctggaaatcacctggtatccatttgacgtggaggacatgaccgcatcctcaggcgctgggaacaaggcagcagcccttcagaggagaatgtccatgtacagccagggtaccccggaaacgcccaccttcaaagaccactccttcttttcaaatctacctgatgacatctttgaaaatggaaaggcagccgaggagaaaatgccactgtcgctcagcttcagtgacctgcccaacggggactgcgccctcacctcccactcaacaggctccccttccaactcaacaaatccagaaattaccatcacccctgcggagtttaacctcagcagcttggcctcccagaatgagggtatggatgacaccagctcagcatcttccaggaactccctgggagaaggccaagagccaaagtcacacctgaaggaggaagacccagaggagcccagaaaacctgcctcggccccatctgaggcttgccgccgacagtcctcaggtgctggggctgagcacctgttccttgagaatgatgttgcagaagcacttctgcaagagtctgaggaggcctctgagctcaagcctgtggaactggacacttcggaaggaaacatcacaaagaggctcacatctgcagaggtgccaatggccacagacaggctgctctctgagggttctgttggtggagaatctgaaggctgcagatcctttctagatggaagcttagaggatgcttttaatgggcttttacttgcattagaaccacataaagagcagtataaagagtttcaggatctgaaccaagaagtcatgaatttggatgatattctaaaaaagtag

SEQ ID NO: 13 - DNA, 21 bp, Artificial Sequence
  primer
ggaaggaaacatcacaaagag

SEQ ID NO: 14 - DNA, 18 bp, Artificial Sequence
  primer
gcagccttcagattctcc

SEQ ID NO: 15 - DNA, 20 bp, Artificial Sequence
  primer
agagtggtgctgaggattgg

SEQ ID NO: 16 - DNA, 20 bp, Artificial Sequence
  primer
ccctcatgctctagcgtgtc

SEQ ID NO: 17 - DNA, 20 bp, Artificial Sequence
  control AON
auagtcttcctgtuguggcu

SEQ ID NO: 18 - DNA, 46 bp, Artificial Sequence
  Fig 1B.
aaggaaacatcacaaagcagctggtcaagaggctcacatctgcaga

SEQ ID NO: 19 - DNA, 3123 bp, Artificial Sequence
  cDNA sequences encoding a C-terminally HA-tagged mutant RIPOR2
atgttggtaggatcccagtctttttcgcctggagggcccaatgggatcattagaagccagtcctttgcgggtttcagcggcctccaggaaaggcgatccaggtgtaactccttcattgaaaattcctccgctctcaagaagcctcaggccaaactgaagaaaatgcacaatttaggccacaaaaacaacaatccccccaaagagcctcagcctaaaagggtggaagaagtctacagggccttgaaaaatggacttgatgaatatctggaggttcaccagacggagctggacaagttgacagctcagttaaaagatatgaaaagaaactctcgcctgggtgtactgtatgacctagacaagcaaattaaaacaattgaaagatacatgagacgcctggagtttcatataagtaaggtagatgaactctatgaagcttattgtatccagcgacgcctccaggatggtgccagcaaaatgaagcaagccttcgcaacatcccctgccagcaaagctgcccgggagagtctgacagagatcaatcggagcttcaaggagtacacagagaatatgtgcaccattgaagtggagctagagaatctgctgggagaattctccatcaagatgaaaggtctggctggctttgcacgcctctgtcctggagatcaatatgaaattttcatgaagtatggccggcagcggtggaaactgaaaggcaaaatagaagtaaatggcaagcagagctgggatggagaagaaacagtttttctgcccctgatagttgggttcatctccatcaaggtcacggagctcaaagggctagcaactcacatcctggtaggtagcgtgacctgtgagaccaaagagctgtttgcagcccgacctcaggtagtggctgtcgacatcaatgaccttggtaccatcaaactgaacctggaaatcacctggtatccatttgacgtggaggacatgaccgcatcctcaggcgctgggaacaaggcagcagcccttcagaggagaatgtccatgtacagccagggtaccccggaaacgcccaccttcaaagaccactccttcttttcaaatctacctgatgacatctttgaaaatggaaaggcagccgaggagaaaatgccactgtcgctcagcttcagtgacctgcccaacggggactgcgccctcacctcccactcaacaggctccccttccaactcaacaaatccagaaattaccatcacccctgcggagtttaacctcagcagcttggcctcccagaatgagggtatggatgacaccagctcagcatcttccaggaactccctgggagaaggccaagagccaaagtcacacctgaaggaggaagacccagaggagcccagaaaacctgcctcggccccatctgaggcttgccgccgacagtcctcaggtgctggggctgagcacctgttccttgagaatgatgttgcagaagcacttctgcaagagtctgaggaggcctctgagctcaagcctgtggaactggacacttcggaaggaaacatcacaaagaggctcacatctgcagaggtgccaatggccacagacaggctgctctctgagggttctgttggtggagaatctgaaggctgcagatcctttctagatggaagcttagaggatgcttttaatgggcttttacttgcattagaaccacataaagagcagtataaagagtttcaggatctgaaccaagaagtcatgaatttggatgatattctaaaatgcaagccagcagtaagccgcagcaggtcttccagtttaagtctcacagttgaaagtgctttagaaagctttgatttcctgaacacctctgattttgacgaggaggaggatggtgatgaggtttgtaatgttggcggaggtgctgactcagtattttcagacactgagactgagaaacacagttacaggtcggttcacccagaagccagggggcatctcagtgaagcgctcactgaagacacaggagttgggaccagtgtggcaggaagtcctctcccactgaccacaggcaacgagagcctggacatcaccatcgtcaggcacctccagtactgcacccaactcgtgcagcaaattgttttctcaagcaaaaccccatttgtggcaagaagtctcttagagaagctttctaggcagatccaagtgatggagaaactcgcagctgtcagtgatgagaacataggaaatatcagttctgttgtggaagccataccagaatttcacaaaaagctgtctttgctgtcattctggaccaagtgctgcagccctgttggtgtctaccacagcccagcggacagagtgatgaagcagctggaggccagctttgccagaactgtcaacaaagaatatccaggacttgcagacccagtgtttcgaaccctggtgtcccaaattctggaccgggctgagcctctgctttcctccagcctgtcctcggaagtcgtcactgttttccagtattacagttacttcaccagccacggcgtcagtgacctggagagttacctgagccagctggccaggcaagtttccatggttcagactctgcaatcactaagagatgaaaaactgctacaaaccatgagtgaccttgctcccagcaacctcctggcccagcaggaagtactcaggactctggctctgctattaaccagagaggacaacgaagttagtgaggctgtgacgctttacttggcagcagcctccaaaaatcagcatttcagggaaaaggccttgctctattactgtgaagcactaacaaagacaaacctccagctccagaaagcagcttgcctggctctgaaaatccttgaggctactgaaagcattaaaatgctggtgacattgtgtcaatctgatactgaagaaatcagaaatgtggcctcagaaaccctcttgtctctgggagaagatgggcggctggcatatgaacaattggacaaatttcctcgagactgtgttaaagtcggaggtcgtcatggaactgaagttgccacagcctttgacccagctttcttgtacaaagtggttcgatctagaatggctagctatccgtatgatgttcctgattatgctagcctctag

SEQ ID NO: 20 - DNA, 3135 bp, Artificial Sequence
  cDNA sequences encoding a C-terminally HA-tagged wildtype RIPOR2
atgttggtaggatcccagtctttttcgcctggagggcccaatgggatcattagaagccagtcctttgcgggtttcagcggcctccaggaaaggcgatccaggtgtaactccttcattgaaaattcctccgctctcaagaagcctcaggccaaactgaagaaaatgcacaatttaggccacaaaaacaacaatccccccaaagagcctcagcctaaaagggtggaagaagtctacagggccttgaaaaatggacttgatgaatatctggaggttcaccagacggagctggacaagttgacagctcagttaaaagatatgaaaagaaactctcgcctgggtgtactgtatgacctagacaagcaaattaaaacaattgaaagatacatgagacgcctggagtttcatataagtaaggtagatgaactctatgaagcttattgtatccagcgacgcctccaggatggtgccagcaaaatgaagcaagccttcgcaacatcccctgccagcaaagctgcccgggagagtctgacagagatcaatcggagcttcaaggagtacacagagaatatgtgcaccattgaagtggagctagagaatctgctgggagaattctccatcaagatgaaaggtctggctggctttgcacgcctctgtcctggagatcaatatgaaattttcatgaagtatggccggcagcggtggaaactgaaaggcaaaatagaagtaaatggcaagcagagctgggatggagaagaaacagtttttctgcccctgatagttgggttcatctccatcaaggtcacggagctcaaagggctagcaactcacatcctggtaggtagcgtgacctgtgagaccaaagagctgtttgcagcccgacctcaggtagtggctgtcgacatcaatgaccttggtaccatcaaactgaacctggaaatcacctggtatccatttgacgtggaggacatgaccgcatcctcaggcgctgggaacaaggcagcagcccttcagaggagaatgtccatgtacagccagggtaccccggaaacgcccaccttcaaagaccactccttcttttcaaatctacctgatgacatctttgaaaatggaaaggcagccgaggagaaaatgccactgtcgctcagcttcagtgacctgcccaacggggactgcgccctcacctcccactcaacaggctccccttccaactcaacaaatccagaaattaccatcacccctgcggagtttaacctcagcagcttggcctcccagaatgagggtatggatgacaccagctcagcatcttccaggaactccctgggagaaggccaagagccaaagtcacacctgaaggaggaagacccagaggagcccagaaaacctgcctcggccccatctgaggcttgccgccgacagtcctcaggtgctggggctgagcacctgttccttgagaatgatgttgcagaagcacttctgcaagagtctgaggaggcctctgagctcaagcctgtggaactggacacttcggaaggaaacatcacaaagcagctggtcaagaggctcacatctgcagaggtgccaatggccacagacaggctgctctctgagggttctgttggtggagaatctgaaggctgcagatcctttctagatggaagcttagaggatgcttttaatgggcttttacttgcattagaaccacataaagagcagtataaagagtttcaggatctgaaccaagaagtcatgaatttggatgatattctaaaatgcaagccagcagtaagccgcagcaggtcttccagtttaagtctcacagttgaaagtgctttagaaagctttgatttcctgaacacctctgattttgacgaggaggaggatggtgatgaggtttgtaatgttggcggaggtgctgactcagtattttcagacactgagactgagaaacacagttacaggtcggttcacccagaagccagggggcatctcagtgaagcgctcactgaagacacaggagttgggaccagtgtggcaggaagtcctctcccactgaccacaggcaacgagagcctggacatcaccatcgtcaggcacctccagtactgcacccaactcgtgcagcaaattgttttctcaagcaaaaccccatttgtggcaagaagtctcttagagaagctttctaggcagatccaagtgatggagaaactcgcagctgtcagtgatgagaacataggaaatatcagttctgttgtggaagccataccagaatttcacaaaaagctgtctttgctgtcattctggaccaagtgctgcagccctgttggtgtctaccacagcccagcggacagagtgatgaagcagctggaggccagctttgccagaactgtcaacaaagaatatccaggacttgcagacccagtgtttcgaaccctggtgtcccaaattctggaccgggctgagcctctgctttcctccagcctgtcctcggaagtcgtcactgttttccagtattacagttacttcaccagccacggcgtcagtgacctggagagttacctgagccagctggccaggcaagtttccatggttcagactctgcaatcactaagagatgaaaaactgctacaaaccatgagtgaccttgctcccagcaacctcctggcccagcaggaagtactcaggactctggctctgctattaaccagagaggacaacgaagttagtgaggctgtgacgctttacttggcagcagcctccaaaaatcagcatttcagggaaaaggccttgctctattactgtgaagcactaacaaagacaaacctccagctccagaaagcagcttgcctggctctgaaaatccttgaggctactgaaagcattaaaatgctggtgacattgtgtcaatctgatactgaagaaatcagaaatgtggcctcagaaaccctcttgtctctgggagaagatgggcggctggcatatgaacaattggacaaatttcctcgagactgtgttaaagtcggaggtcgtcatggaactgaagttgccacagcctttgacccagctttcttgtacaaagtggttcgatctagaatggctagctatccgtatgatgttcctgattatgctagcctctag

SEQ ID NO: 21 - DNA, 20 bp, Artificial Sequence
  AON6-8oxoG
  misc_feature (16)..(16): n is an g modified 8-oxoguanine base
gagcctctttgtgaunuuuc